(19) United States
(12) Patent Application Publication
(10) Pub. No.: US 2004/0097385 A1
(43) Pub. Date: May 20, 2004

(54) Viscoelastic cleansing gel with surfactant solutions containing polysaccharides and their derivatives polysaccharide hydrocolloids

(75) Inventors: Liang Bin Chen, Hoffman Estates, IL (US); Priscilla LaBonville Walling, Darien, IL (US)

Correspondence Address: UNILEVER, PATENT DEPARTMENT, 45 RIVER ROAD, EDGEWATER, NJ, 07020, US

(73) Assignee: Unilever Home & Personal Products USA, Division of Conopco, Inc.

(21) Appl. No.: 10/298,372

(22) Filed: Nov. 18, 2002

(51) Int. Cl. A61K 7/50

(52) U.S. Cl. 510/130; 510/403

(57) ABSTRACT

A viscoelastic cleansing gel composition, comprising:

a. about 1 to about 20% of an anionic surfactant; and

b. 0 to about 20% of an amphoteric surfactant;

c. 0 to about 20% of an nonionic surfactant; and

d. about 0.05 to about 5% of a polysaccharides, or their derivative hydrocolloid gelling agents wherein said composition has a G′ₕ in the range of about 50 to about 2000 Pa.

### BACKGROUND OF THE INVENTION

Often children and infants do not enjoy taking a bath. For many years various types of floating toys have been introduced into the bathtub so as to interest children and infants while they are taking a bath.

It would be desirable to develop other ways of entertaining children and infants while they are taking a bath.

The present invention provides hair shampoos and body wash gels which have a consistency such that they jiggle like gelatin, and yet they can hold a shape. In fact these compositions can be molded into various shapes that are of interest to children and infants. Such shapes may include ducks, fish, birds, dinosaurs, planes, trains, and the like.

Such jiggly and shaped hair shampoos and body wash gels are of interest to children and infants who can play with these hair shampoos and body wash gels and even mold these hair shampoos and body wash gels, themselves, before these compositions dissolve in water and release cleansing surfactant. As such these hair shampoos and body wash gels cause children to better enjoy taking a bath or even a shower. It will of course, also be appreciated that the compositions of the invention may be employed by adults.

The compositions of the invention which can be characterized as gels or semisolids or viscoelastic compositions, can have more appeal than conventional bar soaps, in that the compositions of the invention tend to lather more easily than conventional bar soaps, and also tend to form a richer lather than conventional bar soaps.

The following is a list of patents and patent applications and a product that relate to the field of the invention.

U.S. Pat. No. 6,426,326 discloses to liquid cleansing compositions in lamellar phase which possess a lotion-like appearance conveying signals of enhanced moisturization. However, these liquids often undergo an irreversible decrease in viscosity under freeze/thaw conditions, losing their moisturization signals. The use of low salt levels in amphoteric and anionic surfactants in a structured liquid product has been found to improve its freeze/thaw stability.

WO 01/38475 discloses a product for use in a fabric laundering process which is in the form of a self-supporting aqueous gel and which comprises one or more fabric treating agents, a gelling agent and one or more surfactants comprising a polypeptide or polysaccharide.

EP 0875236 discloses an aqueous composition for treating keratin fibers, especially human hair, which comprises natural and/or synthetic ingredients with a food or pleasurable aroma and a bitter and with a molecular weight of 250 g/mole which is 10 mg/l soluble in the formulation at 20 degrees C.

JUNGLE GEL is a commercial product which comprises:

about 86.4% water;

about 9.1% sodium lauryl ether sulfate; and

about 1.5% PEG pentaerythrityl tetrastearate, as well as fragrances, moisturizing oils, colors, and preservatives.

Canadian Application No. 2,194,442 discloses hydrogels which are used as a suitable application form for using active substances in the treatment of skin injuries and/or for the cosmetic treatment of sensitive sites on the skin and nails. These hydrogels are sheet-like, rigid elastic structures adapted to the contours of human body sites and comprise therapeutic and/or cosmetic active substances.

U.S. Pat. No. 5,965,502 discloses aqueous, viscoelastic surfactant solutions for the cleaning of hair and skin which contain:

(A) from 4 to 25% by weight of an anionic surfactant;

(B) from 0 to 10% by weight of a betainic surfactant;

(C) from 0 to 20% by weight of a nonionic surfactant;

(D) from 0 to 6% by weight of an electrolyte;

(E) from 0 to 5% by weight of a water-soluble polymer; and

(F) from 0 to 5% by weight of a further constituent;

in which the sum of the amounts of (A), (B), and (C) is at least 10% by weight and the sum of the amounts of (C), (D), and (E) is between 2 and 20% by weight, in each case based on the total weight of the aqueous solution, and having a shear modulus, G₀, between 50 and 500 Pa at temperatures between 20 and 40.degree. C. and a pH of from 4 to 8, and in which the conditions for the identity of the storage modulus, G′, and the loss modulus, G″, are in the angular frequency range between 0.1 and 60 rad.multidot.s.sup.-1, exhibit optimum flow behavior for the intended applications.

GB 2,280,906 discloses a shaped toiletry product which comprises a gel comprising a gelling agent, preferably up to 15% gelatin, water and at least one surfactant. The surfactant is retained in the gel and is released on contact with warm water. The use of a gel of suitable composition enables toiletry products that are typically in liquid form, for example, bath gels, shower gels and shampoos to be made in a shaped stable form.

U.S. Pat. No. 6,329,331 discloses an aqueous detergent composition, which is in the form of a thickened, mobile fluid, comprising foaming detergent and a polymer or polymer mixture which is capable of forming a reversible gel, which polymer or mixture is present in the composition as a multiplicity of individual gel particles.

U.S. patent application Ser. No. 10/242,390 filed Sep. 12, 2002 which relates to a viscoelastic cleansing gel composition, comprising:

a. about 4 to about 25% an anionic surfactant; and

b. 0 to about 20% of an amphoteric surfactant;

wherein said composition has a G′ₕ at 63 rad/s in the range of about 100 to about 2000 Pa.;

a G′/G″ or crossover frequency, ω_{c}, in the range of about 0.01 to 10 rad/sec; and

wherein the ratio of anionic to amphoteric surfactant is in the range of about 1:3 to 100:0, is described.

### SUMMARY OF THE INVENTION

The present invention relates to a viscoelastic cleansing gel, comprising:

a. about 1 to about 15% an anionic surfactant; and

b. 0 to about 15% of an amphoteric surfactant;

c. 0 to about 15% of an nonionic surfactant; and

d. about 0.05 to about 5% of a polysaccharides or their derivative hydrocolloid gelling agents wherein said composition has a G′ₕ in the range of about 50 to about 2000 Pa.

The present invention also relates to a method for cleaning the hair or skin which comprises applying to the hair or skin a viscoelastic cleansing gel as described herein.

### DETAILED DESCRIPTION OF THE INVENTION

Unless indicated otherwise, as used herein, “%” means weight %. The starting materials set forth herein are either known or can be prepared in accordance with known methods. The frequency at which G′=G″ or ω_{c} are used interchangeably to mean crossover frequency.

### BRIEF DESCRIPTION OF THE FIGURES

FIG. 1 is a graph of the dynamic oscillatory behavior of a typical micellar cosmetic cleanser that flows like a conventional liquid. The rheological parameter, G′ₕ and ω_{c} are outside the range of the current invention.

FIG. 2 is a graph of the dynamic oscillatory behavior of a viscoelastic cleanser prepared according to claim 1 of the present application. The G′ is larger than the G″ over the frequency range of the measurement and there is no crossover frequency.

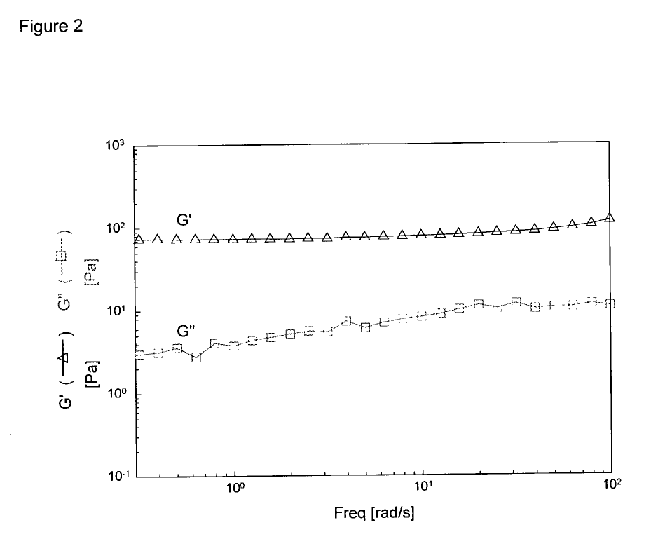

Mixed anionic/amphoteric or anionic/nonionic surfactant solutions are of great importance in a wide range of industrial and consumer products. The flow properties of these systems affect the manufacturing process, package selection and consumer perception of the products. These surfactants, which can organize themselves into different microstructures, may also exhibit viscoelastic properties. For cosmetic cleansers such as currently marketed shampoos and body wash liquids, the surfactant systems are in the normal micellar (L₁) region of the phase diagram. These systems are composed of elongated and rod-like micelles, which exhibit entangled polymer-like flow behavior because of their length and flexibility. The viscoelastic behavior of these cosmetic cleansers can be either described by the Maxwell model or other models that deviate from the Maxwell model (see H. Hoffman and H. Rehage, in Suffactant Solutions, New Methods of Investigation; R. Zana, Ed.; Marcel Dekker: New York, 1987; Chapter 4, pp.209-239). Micellar solutions that behave like Maxwell fluids can be represented by a single shear modulus (G₀) and a single structure relaxation time constant (τ). For those systems that deviate from the Maxwell model, for which some cosmetic cleansers are examples, the viscoelastic response cannot be represented by a single G₀ and a single structural relaxation time τ. Cosmetic cleansers in the micellar phase all exhibit a G′/G″ crossover frequency, ω_{c}, where G′ (elastic modulus) is equal to G″ (loss modulus). Below the crossover frequency, G′ is lower than G″ and above the crossover frequency, G′ is larger than G″ and is approximately equal to G₀. For the Maxwell model, the crossover frequency can be related to the structural relaxation τ as below

τ=1/ω_{c}

and the G′ at the crossover frequency, G′_{c}, can also be related to G₀ as shown below.

G₀=2G′_{c}.

The viscoelastic parameters, G′, G″ and ω_{c} can be determined by using dynamic oscillatory measurement. For the present invention, these measurements were made either by a Bohlin CVOR rheometer with 40 mm cone and plate geometry or a Rheometric ARES rheometer with 25 mm cone and plate geometry. The cone angle and gap spacing for the Bohlin 40 mm cone and plate are 0.6 rad (4 degree) and 0.150 mm, respectively. The Rheometric ARES 25 mm cone and plate has 0.1 radians cone angle and 0.051 mm gap spacing. The measurement is made at 25° C. An angular frequency range of 0.01 to 125 rad/s is applied to the surfactant solutions at 10% strain, which is in the linear viscoelastic region. In order to compare the elastic component of the present invention to other non-Maxwell behavior surfactant systems, G′ at 63 rad/s is recorded. This is valid for a lot of cosmetic cleansers that exhibit Maxwell behavior because the G′ at the high frequency region tends towards the plateau value G₀. For the present invention, G′ measured at 63 rad/s is labeled as G′ₕ.

Most of current micellar phase cosmetic cleansers have G′ₕ smaller than 400 Pa and ω_{c} greater than 10 rad/s. Low elasticity contributions and high ω_{c} allows liquid cleanser to pour out of a container at a reasonable flow rate that is consumer preferred.

However, it has been discovered that when a sufficient amount polysaccharides or their derivative gelling agents such as Carrageenan or agar were dissolublized in the surfactant solution and hot solution is allowed to cool to room temperature, the surfactant solution will set to form a range of gel textures. These gels are stable at room temperature but can be remelted by heating above the gel temperature. On cooling the system will re-gel. The formation of gels by natural polysaccharides arises from the physical interaction between the polymer molecules. Hydrogen bonding is usually the major cause of the interaction. For a detailed description on the formation of gels in these types of polysaccharides see R. L. Whistler and J. N. BeMiller, Industrial Gum: Polysaccharides and their Derivatives, Academic Press, 1993. The gel will possess a strength dependent on the strength of intermolecular bonding. If the bonding is weak, it may be broken and the total gel structure disrupted by mild stirring. In this case, the weak gel is said to be thixotropic. When the intermolecular bonding is strong enough, a more identifiable gel forms that may not be easily broken by stirring. Once the gel is formed it may be strong enough that when under applied stress, the gel will separate or cleave as seen with gelatin gels. In some polymeric systems, once a gel is formed, the polymer molecules can further rearrange themselves by sliding over each other or simply moving together to strengthen the overall physical network structure which will cause a decrease in the water-filled spaces between the molecules. Hence water is exuded from the gel to produce a phenomena which is commonly know as syneresis.

When the viscoelastic properties of these gelled shampoo systems are measured, if G′ is always larger than the G″ over a frequency range of 0.01 to 125 rad/s then is no crossover frequency, ω_{c}. For the present invention, the G′ₕ is set between 50 to 2000 Pa (Preferably from 100 to 600 Pa). At this Theological range, the cleanser behaves like a semi-solid and retains its shape after being dispensed from a container. Cleansers with lower G′ₕ will be too soft to retain its shape. On the other hand cleansers with a higher value of G′ₕ will be perceived to be too hard and difficult to break down and dissolve.

Although xanthan gum can be incorporated as a thickener in aqueous compositions containing surfactant, the resultant products tend to have a stringy texture and a slimy feel. There are a number of other polymers of biological origin that are incapable of forming gels upon cooling. These polymers are either incompatible with the surfactants or the environment that is required for the preparation of the cleanser and therefore prohibit the formation of a gel capable of retaining its shape.

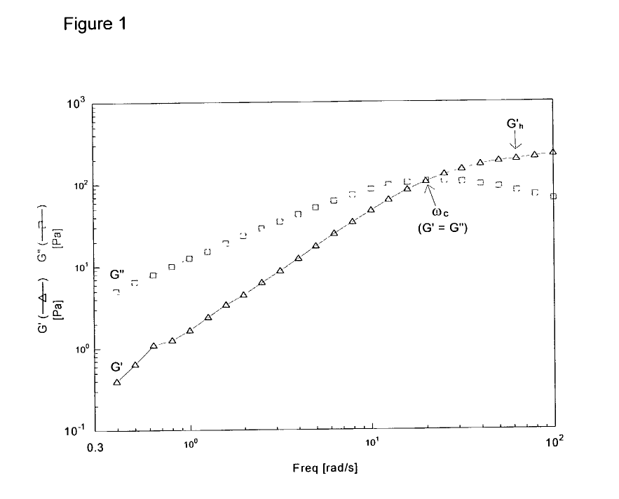

Example 1 is a micellar cosmetic handwashing liquid using an SLS, SLES-2 and cocamidopropyl betaine system that exhibits viscoelastic properties. The viscoelastic profile of this composition is shown in FIG. 1. FIG. 1 shows a G′ₕ lower than 500 Pa and a ω_{c} greater than 10 rad/s, as taught in U.S. Pat. No. 5,965,502. This composition flows like a conventional liquid and not like a gel.

### EXAMPLE 1

| Ingredients | Weight Percent |
|---|---|
| Water | QS |
| Sodium Lauryl Sulfate | 1.7 |
| Sodium Laureth Sulfate (2-mole) | 6.6 |
| Cocamidopropyl Betaine | 2.5 |
| Polyquaternium-10 | 0.15 |
| Methocel 40-100 | 0.15 |
| Sodium Chloride | 1 |
| Add Fragrance, color and preservatives | * |
| Add NaOH or citric acid to adjust pH | * |
| between 5 to 6 | |
| ω_{c} | yes |
| G′ₕ | 199 Pa |

* Added as needed.

Controlling the concentration of the surfactants allows assembly of the surfactants into non-micellar gel phases. Surfactant molecules in solution will assemble into different microstructures. When there is sufficient surfactant to form micelles (concentrations above the critical micelle concentration or CMC), for example, spherical, cylindrical or rod-like micelles may form. As surfactant concentration increases, ordered liquid crystalline phases such as the lamellar phase (see Tiddy, G. J. T. Physics Reports 1980, 57,1-46; Tiddy, G. J. T; Walsh, M. F. In Aggregation Processes in Solution, Wyn-Jones, E.; Gormally, J., Eds., Elsever, Oxford; 1983, Chapter 7.), the hexagonal phase (see Kilpatrick, P. K; Khan, S. A; Tayal, A; Blackburn, J. C. in Structure and Flow in Surfactant Solution, Herb, C. A; Prudhomme, R. K., Eds., ACS Symposium Series 578, ACS, Washington, D.C., 1994, Chapter 15) or the cubic phase (see Rosevear, F. B. J. Soc. Cosmet. Chem. 1968, 19, 581; Gradzielski, M; Hoffman, H. In The Structure, Dynamics and Equilibrium Properties of Colloidal Systems; Bloor, D. M.; Wyn-Jonesm E., Eds.; Kluwer Academic Publishers: 1990, p. 427) may form. The lamellar phase, for example, consists of alternating surfactant bilayers and water layers. These layers are not generally flat but fold to form submicron spherical onion like structures called vesicles or liposomes. The hexagonal phase, on the other hand, consists of long cylindrical micelles arranged in a hexagonal lattice. The present invention relates to compositions with viscoelastic properties that are dominated by the cylindrical or rod-like micellar phase.

U.S. Pat. No. 6,426,326 relates to liquid cleansing compositions in lamellar phase which possess a lotion-like appearance conveying signals of enhanced moisturization. Lamellar phase liquids in oscillating measurements generally have storage modulus (G′) fairly independent of frequency and always larger than the loss modulus (G″).

Example 2 in U.S. Pat. No. 6,426,326 is a structured liquid body wash in the lamellar phase using SLES-3, Cocamide MEA, cocamidopropyl betaine and lauric acid system. This composition does not contain a hydrocolloid gelling agent. The presence of the lauric acid transforms the surfactant solution from a micellar phase into a structured lamellar phase. This type of structured liquid provides another type of rheological behavior that is different from the micellar solution. The viscoelastic profile shows that both the elastic (G′) and the loss (G″) moduli depend much less on the frequency than in the micellar phase. Furthermore G′ is larger than G″ over this frequency range and there is no crossover frequency. These viscoelastic behaviors provide the cleanser a lotion-like appearance yet it cannot retain its shape over a reasonable period of time.

### EXAMPLE 2

| Ingredients | |
|---|---|
| Water | Qs |
| SLES-3 | 12% |
| Cocamide MEA | 2% |
| Cocamidopropyl Betaine | 6% |
| Lauric Acid | 2.8% |
| Moisturizing oil (e.g. sunflower seed | 25% |
| oil, petrolatum or mineral oil) | |
| Add Fragrance, color and | * |
| preservatives | |
| Add NaOH or citric acid to adjust pH | * |
| Between 5 to 6 | |
| ω_{c} | No crossover |
| G′ₕ | 240 Pa |

* Added as needed.

The present invention relates to a hydrocolloid containing surfactant solution compositions having viscoelastic ranges for surfactant solutions necessary to ensure optimum shape retention with good dissolution and foaming properties. The surfactant composition with incorporation of polysaccharide or their derivatives hydrocolloid, together with the rheological parameter G′ₕ are among the distinguishing characteristics of the compositions of the invention.

The viscoelastic properties of the surfactant solution can be enhanced by the incorporation of water soluble polymers which are polysaccharides, or polysaccharide hydrocolloid gelling agents. The most common polymers used in cosmetic cleansers are hydrocolloids which are hydrophilic polymers of vegetable, animal, microbial or synthetic origin (see R. L. Whistler and J. N. BeMiller, Industrial Gum: Polysaccharides and their Derivatives, Academic Press, 1993). These polymers generally contain many hydroxyl groups and may be polyelectrolytes. Examples include but are not limited to agar, carrageenan, polyvinyl alcohol, gellan gum and xanthan gum. Most of the hydrocolloids that are of biological origin, have the ability to form reversible gels which melt when heated but revert to a gel when subsequently cooled. One well known example of a hydrocolloid which forms reversible gel is Carrageenan.

At low hydrocolloid concentration, the viscoelastic properties are dominated by the micellar network. As the concentration of the hydrocolloid increases, the viscoelastic behaviors change from a temporary network within the micellar solution to a physically crosslinked network of a gel. The main parameters that distinguish physically crosslinked network gel systems from micellar solution dominated rheologies are that the elastic modulus (G′) has to be always larger than loss modulus (G″), hence no crossover frequency, ω_{c} in the physically crosslinked network gel system. In the micellar dominated rheology, there is a crossover frequency and a plateau modulus at high frequency, G′ₕ. Below the crossover frequency, G′ is lower than G″ and above the crossover frequency, G′ is larger than G″.

Example 3 is a cleanser product using a recipe similar to example 5 from GB 2280906A. The polymer thickener is gelatin and the resulting G′ is much larger than G″ and there is no crossover frequency from 0.01 to 100 rad/s. This indicates the gel network form of this composition is more permanent or dominant than the micellar network. However, the G′ₕ is high, which make this formulation too difficult to breakdown and dissolve as a cleanser.

### EXAMPLE 3

| Ingredients | |
|---|---|
| Water | q.s. |
| Gelatin (Bloom 250) | 5% |
| SLES-2 | 15.3% |
| Cocamide MEA | 5% |
| Glycerine | 10% |
| ω_{c} | No crossover |
| G′ₕ | 3315 Pa |

During the cooling process if the polysaccharide surfactant solutions are subjected to shear either during or after the gelation process, the application of shear will disrupt normal gelation and result in a “fluid gel” that is pourable and cannot hold a shape (see G. O. Phillips and P. A. Williams, Handbook of hydrocolloids, CRC Press, Woodhead Publishing Limited, Cambridge, England, 2000). U.S. Pat. No. 6,329,331 is an invention that utilizes this type of “fluid gel” liquid. Instead of a gelling network consisting of the polymer a three dimensional physical crosslinked polymer network, throughout the products, the cleanser is a mobile fluid that consists of individual gel particles and inability to retain shape.

The present invention provides hair shampoos or body wash gels which have a consistency such that they jiggle like gelatin, and yet they can hold a shape. In fact these compositions can be molded into various shapes that are of interest to children and infants. Such shapes as indicated above, may include ducks, fish, birds, dinosaurs, planes, trains, and the like.

Such jiggly and shaped hair shampoos and body wash gels are of interest to children and since children and infants can play with these hair shampoos and body gels and even mold these hair shampoos and body gels before the gels dissolve in water and release cleansing surfactant. As such these hair shampoos and body gels cause children to better enjoy taking a bath or even a shower. It will of course, also be appreciated that the compositions of the invention may be employed by adults.

It will further be appreciated that the gels or semisolids or viscoelastic compositions of the invention can have more appeal than conventional bar soaps, in that the compositions of the invention tend to lather more easily than conventional bar soaps, and also tend to form a richer lather than conventional bar soaps.

What now follows is a detailed description of each ingredient which may be included in the compositions of the present invention.

Anionic Surfactants

Suitable anionic surfactants are the alkyl sulfates, alkyl ether sulfates, alkaryl sulfonates, alkaryl isethionates, alkyl succinates, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be saturated and/or unsaturated. The alkyl ether sulfates, alkyl ether phosphates and alkyl ether carboxylates may contain from 1 to 10 ethylene oxide (EO) or propylene oxide (PO) units per molecule, and preferably contain 1 to 3 ethylene oxide units per molecule. Other suitable anionic surfactants include sodium oleyl succinate, amidosulfur succinate, ammonium lauryl sulfosuccinate, ammonium lauryl sulfate, sodium dodecylbenzene sulfonate, triethanolamine dodecylbenzene sulfonate, sodium cocoyl isethionate, sodium lauroyl isethionate and sodium N-lauryl sarcosinate. The most preferred anionic surfactants are sodium lauryl sulfate [SLS], ammonium lauryl sulfate [ALS], sodium lauryl ether sulfate with 1 EO, 2EO and 3EO [SL(EO)₁₋₃S] and ammonium lauryl ether sulfate with 1 EO, 2EO and 3EO [AL(EO)₁₋₃S].

Nonionic Surfactants

The nonionic surfactants suitable for use in the compositions of the invention may include condensation products of aliphatic (C8-C18) primary or secondary linear or branched chain alcohols, phenols, esters, acids and amines. Other suitable nonionics include mono or dialkyl alkanolamides or alkyl polyglucosides. Examples include coco mono or diethanolamide, coco mono isopropanolamide, and coco di glucoside.

The cleansing gels of the invention may include a nonionic surfactant selected from the group consisting of PEG 150 Distearate, cocamide mea, cocamide DEA, and mixtures thereof.

The nonionic surfactant may be selected from the group consisting of

a.

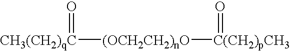

where n has values from 6 to 200, q and p each independently have values of 10 to 18;

(Nonionic surfactants can include molecular structures similar to a) above such as PEG Dilaurate, PEG Dioleate, PEG Dipalmitate, PEG Ditallate.)

b.

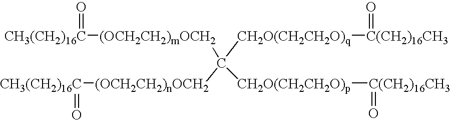

where m+n+q+p=150;

C.

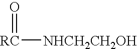

where R could represent the fatty group derived from coconut oil or CH₃(CH₂)ₙ where n has values of 6 to 16, preferably 10;

d. (It could also has structure similar to Cocamide DEA):

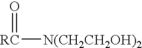

where R could represents the fatty group derived from coconut oil or CH₃(CH₂)ₙ where n has values of 6 to 16, preferably 10;

e. or mixtures thereof.

Amphoteric Surfactants

The amphoteric surfactants suitable for use in the compositions of the invention may include alkyl amine oxides, alkyl betaines, alkyl amidopropyl betaines, alkyl sulfobetaines, alkyl glycinates, alkyl carboxy glycinates, alkyl ampho propionates, alkyl amidopropyl hydroxysultaines, acyl taurates and acyl glutamates wherein the alkyl and the acyl groups have from 8 to 18 carbon atoms. Examples include lauryl amine oxide, cocodimethyl sulfopropyl betaine and preferably lauryl betaine, cocamidopropyl betaine (CAPB), amphoacetate, cocamidopropyl hydroxysultaine and sodium cocamphopropionate.

The cleansing gels of the invention can have an amphoteric surfactant selected from the group consisting of cocamidopropyl betaine and cocamidopropyl hydroxysultaine.

Polysaccharides and Polysaccharide Hydrocolloids

In the present invention, water soluble polymer gelling agents, such as polysaccharides or their derivative hydrocolloids, are needed to physically transform the surfactant dominated solution structure to a physical crosslinked gel structure. Incorporation of these polysaccharide hydrocolloids will change the viscoelastic behavior from a temporary network of a micellar solution to a physically crosslinked network of a gel. Hydrocolloid gelling agents are hydrophilic polymers, of vegetable, animal, microbial or synthetic origin, that generally contain many hydroxyl groups and may be polyelectrolytes. Examples of hydrocolloid gelling agents are polysaccharides or their derivatives such as carrageenan, agar, gellan gum, locust bean gum, pectin, amidated pectin, alginate and processed euchema seaweed gum.

Polysaccharide hydrocolloid used in this current invention can be in the range of about 0.05 to 5% by wt. (preferably about 0.5 to 2). A suitable polysaccharide hydrocolloid gelling agent for this invention is iota carrageenan. Carrageenans are a class of polysaccharides which occur in some red seaweed species. They are linear polysaccharides made up from alternating β-1,3- and α-1,4-linked galactose residues. The 1,4-linked residues are the D-enantiomer and sometimes occur as the 3,6-anhydride. Many of the galactose residues are sulphated.

There are a number of different carrageenan structures and some of these are commercially available. The variations between these structures are dependant on the source of the carrageenan and treatment of it after extraction. See P. Harris, Food Gels, Elsevier, 1990, for a detailed description of different carrageenan types.

Iota carrageenan is sulphated on both the 1,3-linked galactose residues and 1,4-linked residues, while the kappa carrageenan is sulphated on the 1,3-linked galactose residues and not on the 1,4-linked residues. Lambda carrageenan has two sulphate groups on the 1,4-linked residues and one sulphate group on 70% of the 1,3-linked residues.

When preparing the cleansing gel with iota carrageenan, the resulting gel is a soft in texture yet able to retain its shape. Upon application of shear and dilution with water during normal washing action, the cleansing gel dissolves rapidly and forms copious amounts of foam. Kappa carrageenan tends to form a harder textured gels and upon application, the gels tend to fracture and break more like a gelatin. In compositions of the present invention, it is preferable to use iota carrageenan or mixtures of iota and kappa carrageenan.

Lambda carrageen by itself does not form gels because higher charge density prevents the physical crosslinking of the molecules. However it is possible to use Lambda in combination with iota to form gels which are compositions of the invention. Commercially, lambda carrageenan may be present as an impurity in the kappa or iota carrageenan.

Another polysaccharide hydrocolloid gelling agent that may be used in compositions of the invention is agar. Also known as Agar-Agar, it is a strongly gelling hydrocolloid from Maine algae. The term agar covers a family of polymers containing agarose and/or agaropectin. Agarose is a linear polysaccharide, basically made up from β-1,3 galactose residues alternating with α-1,4 galactose residues. The α-1,4 galactose residues are present as the 3,6-anhydride and are the L-enantiomer. The agaropectin has β-1,3 galactose residues alternating with α-1-4,L-galactose residues, but includes sulphate, pyruvate and/or glucuronic acid residues. See P. Harris, Food Gels, Elsevier, 1990, for detail description of agar.

Another polymer that may be used in compositions of the invention is furcellaran. Furcellaran is similar to kappa carrageenan, but is only partially sulphated on the 1,3-linked galactose residues.

Another polymer that may be used in compositions of the invention is gellan gum. Gellan gum is an extracellular polysaccharide secreted by the micro-organism Sphingomonas elodea previously referred to as Pseudomonas elodea. Gellan gum forms gels at low concentrations when hot solutions are cooled in the presence of gel promoting cations. It is available commercially in a substituted or unsubstituted form. The gel properties depend on the degree of substitution with the acyl groups. Low acyl gellan gum produces a gel that is firm and brittle while high acyl gellum gum resulting in a soft and elastic gel.

Synergistic combinations of two different types of polysaccharides can also be used to make shape retaining soft cleanser gel which are compositions of the invention. For example hot solutions of kappa carrageenan and locust bean gum form strong elastic gels with low syneresis when cooled below 50-60° C. Locust bean gum is a galactomannan with a sequences of mannose residues in its polymer chain.

Electrolytes

The compositions of the invention may further comprise an electrolyte in a concentration range of about 0.01 to 5% by wt. Suitable electrolytes are salts such as sodium chloride and ammonium chloride but can also be magnesium chloride, sodium sulfate and also alkali metal salts of carboxylic acids such as sodium citrate.

Water

Water is also included in the compositions of the present invention.

The compositions of the present invention can come in individual shapes and sizes. Each individual shape can be molded and packaged into similarly shaped plastic cups. A number of such cups may be adhered to a piece of cardboard, and sold together on that cardboard. Shapes packaged in plastic cups may also be packed inside a box, for example. Compositions of the invention may also be packaged in a can or tub, and the consumer may be provided with scoops or spoons of varying shapes and sizes which can be used to remove compositions of the invention from the can or tub. Compositions of the invention can be molded in the hands by the child or infant who is using it. Compositions of the invention may also be dissolved in water to form a thick and rich lather for the skin and the hair. Compositions can also come in different colors so as to interest children and infants. Compositions can also contain glitter, pearslescing agents, beads, and small toys so as to interest children and infants. Compositions of the invention of course dissolve in the bath water so that when beads and small toys are included in said compositions. These beads and small toys remain with the child or infant after dissolution of the gel. The infant or child can then play with the toys or beads that remain. Compositions of the invention can also contain an amount of a harmless but bitter tasting ingredient, such as about 0.1 to about 0.2% bitrex, so as to prevent children and infants from eating the compositions of the invention.

### EXAMPLES

Examples 1, 2, 4 to 8 are not compositions of the invention but instead are seven examples of currently marketed shampoos, shower gel, hand wash liquid and body wash liquids that have composition and viscoelastic properties outside those of compositions and the viscoelastic properties of the present invention. Examples 1 and 2 are the hand wash liquid and the body wash gel that were mentioned above, respectively. The structured liquid body wash gel of Example 2 has no crossover of G′ and G″ at frequency range of 0.01 to 100 rad/s. The composition does not contain a hydrocolloid gelling agent and is pourable as a liquid. The kid shampoo has a G′ smaller than G″ over the frequency range of 0.01 to 125 rad/s and the crossover frequency that is too large to be measured by the rheometer. For the rest of the compositions, their Theological properties are out of the ranges specified for the compositions of the present invention. They all appear to be very flowable.

Example 4 is a currently marketed micellar phase shower gel using Ammonium Lauryl/Laureth sulfate, cocamide MEA and cocamidopropyl betaine.

### EXAMPLE 4

| Ingredients | Weight % |
|---|---|
| Water | Qs |
| Ammonium Lauryl Sulfate | 3.8 |
| Ammonium Laureth Sulfate | 2.7 |
| Cocamide MEA | 0.65 |
| PEG-5 Cocamide | 0.35 |
| Cocamidopropyl Betaine | 0.85 |
| Carbomer | 0.4 |
| Add Fragrance, Color and Preservatives | * |
| Add NaOH or Citric Acid to Adjust pH | * |
| G′/G″ Crossover Frequency | yes |
| G′ₕ | 263 Pa |

* Added as needed.

Examples 5 and 6 are two currently marketed micellar phase body wash liquids using SLS, SLES-2 and cocamidopropyl betaine.

### EXAMPLES 5 AND 6

| Ingredients | Weight % | Weight % |
|---|---|---|
| Water | QS | QS |
| Sodium Lauryl Sulfate | 2.7 | 2.7 |
| Sodium Laureth Sulfate (2-mole) | 9 | 9 |
| Cocamidopropyl Betaine | 4 | 4 |
| Polyquaternium-10 | 0.1 | 0.27 |
| Methocel 40-100 | 0.15 | 0.15 |
| Sodium Chloride | 1 | 0.6 |
| Add Fragrance, Color and Preservatives | * | * |
| Add NaOH or Citric Acid to Adjust pH | * | * |
| G′/G″ Crossover Frequency | yes | yes |
| G′ₕ | 299 Pa | 198 Pa |

* Added as needed.

Example 7 is a currently marketed micellar phase shampoo liquid using Ammonium Lauryl/Laureth Sulfate, Cocamide MEA system.

### EXAMPLE 7

| Ingredients | Weight % |
|---|---|
| Water | QS |
| Ammonium Lauryl Sulfate | 5 |
| Ammonium Laureth Sulfate | 4 |
| Cocamide MEA | 1 |
| PEG-5 Cocamide | 0.5 |
| Ammonium Chloride | 1.2 |
| Add Fragrance, Color and Preservatives | * |
| Add NaOH or Citric Acid to Adjust pH | * |
| G′/G″ Crossover Frequency | yes |
| G′ₕ | 130 Pa |

* Added as needed.

Example 8 is a currently marketed micellar phase kids' shampoo liquid using Sodium Trideceth Sulfate, cocamidopropyl hydroxysultaine and disodium lauroamphodiacetate. The crossover frequency is outside the frequency range and the elastic (G′) is smaller than loss (G″) modulus.

### EXAMPLE 8

| Ingredients | Weight % |
|---|---|
| Water | QS |
| Sodium Trideceth Sulfate | 19 |
| Cocamidopropyl Hydroxysultaine | 6 |
| Disodium Lauroamphodiacetate | 5 |
| PEG-80 Sorbitan Laurate | 9 |
| PEG-150 Distearate | 1.5 |
| Sodium Laureth-13 Carboxylate | 1 |
| Polyquaternium-10 | .2 |
| Add Fragrance, Color and Preservatives | * |
| Add NaOH or Citric Acid to Adjust pH | * |
| G′/G″ Crossover Frequency | Too large to be |
| | measured |
| G′ₕ | 58 Pa |

* Added as needed.

The final pH of examples 1, 2, 4 to 8 were adjusted to between 5.5 to 6.

Table 1 lists most of well known shampoo the currently sold in the market. It shows that none of the shampoos have both G′ₕ and cwithin the range defined by the current specification. For those cleanser products that have ω_{c} out the range of the measurement, the elastic (G′) modulus is lower than loss (G″) modulus. Consequently, they all pour out of a container at a reasonable flow rate that is consumer preferred for conventional shampoos. On the other hand these products do not exhibit the “jiggle” behaviors or hold a shape, which are properties shown by the compositions of the present invention.

TABLE 1
Shampoos Currently Sold in United States

| Shampoo Name | ω_{c} | G′ₕ (Pa) |
|---|---|---|
| Pantene Pro-V Clarifying Shampoo | Yes | 175 |
| Pantene Pro-V 2-in-1 shampoo + conditioner | Yes | 223 |
| Pantene Pro-V shampoo | Yes | 215 |
| VO5 normal shampoo | Yes | 125 |
| VO5 moisturizing shampoo | Yes | 128 |
| L'Oreal Vive 2 in 1 shampoo and | Yes | 168 |
| conditioner | | |
| L'Oreal Vive curl-moisture shampoo | Yes | 160 |
| Clairol Herbal Essences shampoo | Yes | 161 |
| Pert plus 2-in-1 shampoo + light conditioner | Yes | 237 |
| Physique amplifying shampoo | Yes | 185 |
| TRESemme Hydrology intense moisture | Yes | 196 |
| shampoo | | |
| Prell rinse clean shampoo | Yes | 354 |
| Thermasilk Heat Activated Regular | Yes | 296 |
| shampoo | | |
| Finesse Plus Shampoo and Conditioner | Yes | 206 |
| Head and Shoulders dandruff shampoo | Yes | 209 |
| Johnson's baby shampoo w/detangling | Greater than 100 | 54 |
| formula | | |
| L'Oreal Kids swim and sport shampoo | Greater than 100 | 28 |
| L'Oreal Kids extra gentle 2-in-1 shampoo | Greater than 100 | 15 |

Preparation of Viscoelastic Surfactant Solution with Polysaccharide Hydrocolloid Gelling Agent:

Compositions of the invention, which are those that jiggle, are prepared by mixing the components in the amounts indicated in the tables below. Water is first added in the mixing chamber. Next the polysaccharide hydrocolloid polymer is dispersed slowly. Once the gelling agent is dispersed, the dispersion is heated to between 75 to 85° C. If an amphoteric or nonionic is to be included in the compositions, it is added next. Next the anionic surfactant is added. Electrolytes can also be added at the end of the process if required. The pH of the solution is adjusted to be between 4.5 to 6.5 depending on the type of polymer and the surfactants used.

The compositions of the invention may be prepared using known starting materials or starting materials which may be obtained by known methods. These compositions may be prepared by methods which are known in the art or which are analogous to those known in the art.

Compositions of the invention can form a gel and can retain a shape. Compositions of the invention can be spread upon rubbing on the hair or skin and dissolve quickly upon dilution with water.

Examples below show how one can make cleanser gels by incorporating polysaccharide hydrocolloid gelling agents into surfactant solution. Using the appropriate gelling agent together with the appropriate anionic, nonionic or amphoteric surfactant concentration can result in a cleanser gel that has the right rheological parameters to exhibit “jiggle” behaviors, to be able to retain a shape and to be able spread, and to be able to dissolve easily upon rubbing and diluting with water.

Examples 9 to 11 show the effect of different types of carrageenan on a cleanser compositions based on Sodium Laureth (n=>3) Sulfate (SLES-3), Cocamidopropyl Betain (CAPB) and water. The carrageenans used in these examples were kappa, Iota, and lambda and the trade names are Gelcain GP 812, Gelcain GP 359, and Gelcain GP 109, respectively. The three carrageenans are supplied by FMC.

| | Example 9 | Example 10 | Example 11 |
|---|---|---|---|
| Ingredients | Wt % | Wt % | Wt % |
| Water | QS | QS | QS |
| Kappa Carrageenan | 2 | — | — |
| Iota Carrageenan | — | 2 | — |
| Lambda Carrageenan | — | — | 2 |
| Sodium Laureth Sulfate | 3.33 | 3.33 | 3.33 |
| (3-mole) | | | |
| Cocamidopropyl Betaine | 2.67 | 2.67 | 2.67 |
| (CAPB) | | | |
| G′ₕ | 1300 Pa | 260 Pa | — |

A comparison of examples 9 to 11 shows that cleanser compositions containing kappa carrageenan form a harder and more brittle gel and has a G′ₕ of about 1300 Pa. The cleanser gel that contains the iota carrageenan gives a softer and shape retaining gel. Upon application of shear and contact with water, this gel breaks easily and readily forms a rich and thick lather. The G′ₕ for the iota carrageenan cleanser gel is 260 Pa, which is much lower than the Kappa carrageenan cleanser gel. Example 11 does not form shape retaining gel.

Examples 12, 13 and 14 illustrate the effect of surfactant concentration on the elastic modulus or the G′ₕ of cleanser gels containing 2 weight percent of iota carrageenan. The surfactants in these examples were used in a constant ration of 5.55 parts SLES-3 to 4.45 parts CAPB. In this study 0.5 weight percent of ammonium chloride is also added.

| | Example 12 | Example 13 | Example 14 |
|---|---|---|---|
| Ingredients | Wt % | Wt % | Wt % |
| Water | QS | QS | QS |
| Iota Carrageenan | 2 | 2 | 2 |
| Sodium Laureth Sulfate | 1.67 | 3.33 | 6.66 |
| (3-mole) | | | |
| Cocamidopropyl Betaine | 1.33 | 2.67 | 5.34 |
| Ammonium chloride | 0.5 | 0.5 | 0.5 |
| G′ₕ | 420 Pa | 170 Pa | 174 Pa |

These examples show that the G′ₕ modulus is decreased from examples 12 to example 14. Even though the G′ₕ for examples 13 and 14 are almost the same. Example 14 with high amount of surfactants exhibits syneresis after 96 hours. It also appears to be more stringy than examples 12 and 13. Comparison of example 10 to example 13 demonstrates that by incorporating 0.5 weight percent of ammonium chloride, the G′ₕ decreases slightly.

Examples 15, 16 and 17 shows the effect of iota carrageenan concentrations on the cleanser gel compositions of the invention. The anionic surfactant in these examples is sodium Laureth sulfate (2-mole).

| | Example 15 | Example 16 | Example 17 |
|---|---|---|---|
| Ingredients | Wt % | Wt % | Wt % |
| Water | QS | QS | QS |
| Iota Carrageenan | 1 | 2 | 4 |
| Sodium Laureth Sulfate | 3.33 | 3.33 | 3.33 |
| (2-mole) | | | |
| Cocamidopropyl Betaine | 2.67 | 2.67 | 2.67 |
| G′ₕ | 98 Pa | 460 Pa | 1190 Pa |

Comparison of examples 15 to 17 show that as the iota carrageenan concentration increases, the modulus of the cleanser gel also increases. Comparison of example 10 to example 16 shows that cleanser gel made from Sodium Laureth Sulfate (2-mole) has higher G′ₕ compared to the Sodium Laureth Sulfate (2-mole) cleanser gel. This suggests that as the ethoxylation of the anionic surfactant increases, the G′ₕ decreases.

Examples 18, 19 and 20 below are cosmetic cleansers of the invention that contain polysaccharide hydrocolloid gelling agents and have G′ₕ within the required Theological ranges and therefore retain shape after being dispensing from containers.

### EXAMPLE 18

| | Weight |
|---|---|
| Ingredients | Percent |
| DI Water | 92.04 |
| Versene 100 | 0.05 |
| Iota Carrageenan | 2 |
| SLES-2 | 2.7 |
| CAPB | 2.2 |
| DMDM Hydantoin | 0.1 |
| Dimethicone | 0.5 |
| Fragrance | 0.4 |
| Color (1% dye solution) | 0.01 |
| Gₕ′ | 280 Pa |

### EXAMPLE19

| | Weight |
|---|---|
| Ingredients | Percent |
| DI Water | 91.04 |
| Versene 100 | 0.07 |
| Guar Hydroxypropltrimonium Chloride | 0.07 |
| Iota Carrageenan | 2 |
| PEG-150 Distearate | 0.37 |
| CAPB | 1.33 |
| Cacoamidopropyl Hydroxysltaine | 1.33 |
| SLES-3 | 3.33 |
| DMDM Hydantoin | 0.05 |
| Fragrance | 0.4 |
| Color (1% dye solution) | 0.01 |
| Gₕ′ | 740 Pa |

### EXAMPLE 20

| Ingredients | Weight % |
|---|---|
| Water | 94.13 |
| Iota Carrageenan | 1.6 |
| Kappa Carrageenan | 0.3 |
| Ammonium Lauryl Sulfate | 1.68 |
| Ammonium Laureth Sulfate | 1.32 |
| Cocamide MEA | 0.34 |
| PEG-5 Cacamide | 0.17 |
| DMDM Hydantoin | 0.05 |
| Fragrance | 0.4 |
| Color (1% dye solution) | 0.01 |
| G′ₕ | 290 Pa |

From the foregoing, it will be appreciated that although specific embodiments of the invention have been described herein for purposes of illustration, various modifications may be made without deviating from the spirit or scope of the invention.

What is claimed is:

1. A viscoelastic cleansing gel composition, comprising:
a. about 1 to about 15% of an anionic surfactant; and
b. 0 to about 15% of an amphoteric surfactant;
c. 0 to about 15% of an nonionic surfactant;
d. about 0.05 to about 5% of a polysaccharides or their derivative hydrocolloid gelling agents wherein said composition has a G′ₕ in the range of about 50 to about 2000 Pa.

2. A composition according to claim 1 which comprises about 1% to about 10% anionic surfactant.

3. A composition according to claim 1 which comprises about 2% to about 5% anionic surfactant

4. A composition according to claim 1 which comprises about 0.01% to about 5% amphoteric surfactant.

5. A composition according to claim 1 which comprises about 0.5% to about 3% amphoteric surfactant.

6. A composition according to claim 1 which comprises about 0.01% to about 5% of a nonionic surfactant.

7. A composition according to claim 1 which comprises about 0.5% to about 3% of a nonionic surfactant.

8. A composition according to claim 1 which comprises about 0.5 to about 3% of a polysaccharide or its derivative hydrocolloid gelling agent.

9. A composition according to claim 1 which comprises about 1 to about 2% of a polysaccharide or its derivative hydrocolloid gelling agent.

10. A composition according to claim 1 wherein said of a polysaccharide gelling agent is carrageenan.

11. A composition according to claim 1 wherein said a polysaccharide gelling agent is iota carrageenan.

12. A composition according to claim 1 wherein said of a polysaccharide gelling agent is a mixture of iota and kappa carrageenan.

13. A composition according to claim 1 which has a G′ₕ in the range of about 200 to about 800 Pa.

14. G′ is always larger than G″ over a frequency range of 0.01 to 125 rad/s and there is no crossover frequency, coc.

15. A composition according to claim 1 wherein said nonionic surfactant is selected from the group consisting of
a. 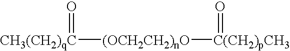
where n has values from 6 to 200, q and p have values of 10 to 18.
b. 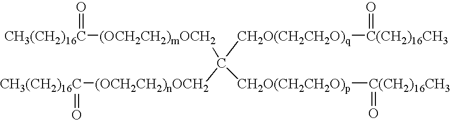
where m+n+q+p=150;
c. 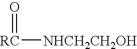
where R could represent the fatty group derived from coconut oil or CH₃(CH₂)ₙ where n has values of 6 to 16;
d. 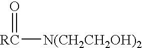
where R could represents the fatty group derived from coconut oil or CH₃(CH₂)ₙ where n has values of 6 to 16;
e. and mixtures thereof.

14. A composition according to claim 13, the value of q in component c is 10.

15. A composition according to claim 13, the value of q in component d is 10.

16. A composition according to claim 1 wherein said nonionic surfactant is selected from the group consisting of PEG 150 distearate, cocamide DEA, and Cocoamide MEA and mixtures thereof.

17. A composition according to claim 1 wherein said anionic surfactant is selected from the group consisting of sodium lauryl sulfate [SLS], ammonium lauryl sulfate [ALS], sodium lauryl ether sulfate with 1 EO, 2EO and 3EO [SL(EO)₁₋₃S] and ammonium lauryl ether sulfate with 1 EO, 2EO and 3EO [AL(EO)₁₋₃S], amido sulfur succinate, and mixtures thereof.

18. A composition according to claim 1 wherein said amphoteric surfactant is selected from the group consisting of, lauryl betaine, cocamidopropyl betaine (CAPB), amphoacetate, cocamidopropyl hydroxysultaine, sodium cocamphopropionate. and mixtures thereof.

19. A composition according to claim 1 which further comprises emollients, anti dandruff agents, skin feel agents, hair dyes, styling polymer, silicone oil, cationic polymers and mixtures thereof.

20. A composition according to claim 1 which further comprises a member selected from the group consisting of a glittering agent, a pearlescent agent, a toy, a bead, and mixtures thereof.

21. A composition according to claim 1 which further comprises occlusive emollients selected from the group consisting of triglyceride oil, petroleum oil, a non-occlusive emollient, selected from the group consisting of glycerin, polyols, and a member selected from the group consisting of cationic polymers and mixtures thereof.

22. A composition according to claim 1 which further comprises an electrolyte.

23. A composition according to claim 22 wherein said electrolyte is selected from the group consisting of ammonium chloride, sodium chloride, potassium chloride and calcium chloride.

24. A composition according to claim 22 wherein said electrolyte is present at about 0.01 to 5%.

25. A composition according to claim 1 wherein said polysaccharide hydrocolloid gelling agent is agar.

26. A composition according to claim 1 which further comprises a thickening agent.

27. A composition according to claim 26 wherein said thickening agent is xanthan gum.

28. A composition according to claim 26 wherein said thickening agent is gelatin.

29. A composition according to claim 1 wherein said anionic surfactant is selected from the group consisting of alkyl sulfates, alkyl ether sulfates, alkaryl sulfonates, alkaryl isethionates, alkyl succinate, alkyl sulfosuccinates, N-alkoyl sarcosinates, alkyl phosphates, alkyl ether phosphates, alkyl ether carboxylates, and alpha-olefin sulfonates, sodium oleyl succinate, ammonium lauryl sulfosuccinate, ammonium lauryl sulfate, sodium dodecylbenzene sulfonate, triethanolamine dodecylbenzene sulfonate, sodium cocoyl isethionate, sodium lauroyl isethionate and sodium N-lauryl sarcosinate.

30. A composition according to claim 1 wherein said anionic surfactant is selected from the group consisting of sodium lauryl sulfate, ammonium lauryl sulfate, sodium lauryl ether sulfate with 1 EO, 2EO and 3EO and ammonium lauryl ether sulfate with 1 EO, 2EO and 3EO, and mixtures thereof.

31. A composition according to claim 1 wherein said nonionic surfactant is selected from the group consisting of condensation products of aliphatic (C₈-C₁₈) primary or secondary linear or branched chain alcohols, phenols, esters, acids and amines. mono or dialkyl alkanolamides and alkyl polyglucosides, and mixtures thereof.

32. A composition according to claim 1 wherein said amphoteric surfactant is selected from the group consisting of alkyl amine oxides, alkyl betaines, alkyl amidopropyl betaines, alkyl sulfobetaines, alkyl glycinates, alkyl carboxy glycinates, alkyl ampho propionates, alkyl amidopropyl hydroxysultaines, acyl taurates and acyl.

33. A method for washing hair which comprises applying to the hair a composition according to claim 1.

34. A method for washing skin which comprises applying to the skin a composition according to claim 1.

35. A composition according to claim 1 wherein the cosmetic cleanser holds it shape upon dispensing, and spreads onto skin or hair.

36. A composition according to claim 1 wherein the surfactants are in the normal micellar (L₁) region of the phase.
